(19) United States
(12) Patent Application Publication
(10) Pub. No.: US 2009/0312663 A1
(43) Pub. Date: Dec. 17, 2009

(54) System and Method for Neurometric Analysis

(75) Inventors: Erwin R. John, Mamaroneck, NY (US); Leslie S. Prichep, Mamaroneck, NY (US); Robert Isenhart, Brooklyn, NY (US); David Cantor, Alpharetta, GA (US)

Correspondence Address: FAY KAPLUN & MARCIN, LLP, 150 BROADWAY, SUITE 702, NEW YORK, NY, 10038, US

(73) Assignee: New York University, New York, NY (US)

(21) Appl. No.: 12/139,228

(22) Filed: Jun. 13, 2008

(51) Int. Cl. A61B 5/0476; G06Q 50/00

(52) U.S. Cl. 600/544; 705/3

(57) ABSTRACT

A system, comprises a plurality of neurometric analyzing software modules operating on electroencephalogram (EEG) data, each module performing a defined task with respect to neurometric analysis of the EEG data and a database including normative neurometric data in combination with a server including an authorization module, the authorization module comparing data received from a remote user to determine whether the user is authorized to utilize any of the plurality of analyzing software modules and an analysis module configured to receive input data from a patient and select one of the neurometric analyzing modules based on the input data, wherein the selected one of the neurometric analyzing modules compares the input data to the normative neurometric data to generate an output, the analysis module selecting the one of neurometric analyzing modules from a set of subscribed neurometric analyzing modules.

### BACKGROUND INFORMATION

The human nervous system exhibits complex electrical and magnetic activities which may be observed and recorded. At the present time it is known that faint electrical and magnetic waves in the human central nervous system, which includes the brain and spine, may be detected and analyzed by non-invasive methods. One way to detect and record neurological activity in the central nervous system is by electroencephalography which is widely used in clinical practice and is commonly found in neurological clinics and hospitals.

An electroencephalograph (EEG) is a device which measures and records neurological activity by detecting the difference in electrical potential between electrodes placed onto various sites on or near the patient's head, such as the scalp, cortex and/or cerebrum. Each EEG channel corresponds to a particular electrode combination attached to the patient. The sensed EEG potential at each channel is amplified by a differential amplifier, and the amplified output is recorded. The output may be recorded in analog or digital form. If recorded digitally, the waves may be quantified and representative values may be extracted. Nonetheless, typically, a neurologist must evaluate the EEG record to determine abnormalities in the EEG waveforms.

Neurometric Analysis is the objective statistical evaluation of the numerical values extracted from neurological signals emitted by the central nervous system relative to a normative and/or clinical database representative of the population and extending across the whole human life span. The neurometric method is an automatic computerized method for evaluating brain functions. A number of successful commercial instruments have been produced based upon the computer-aided neurometric method. One of which is the “Spectrum 32” by Cadwell Laboratories (Kennewick, Wash.). However, the “Spectrum 32” is large, non-portable and relatively expensive to purchase and maintain.

Neurological signals emitted by the central nervous system exhibit systematic changes as one develops and ages. These systematic changes may be extrapolated from a pool of normative data. Based on these identified systematic changes, patients who exhibit neurological signals that significantly deviate from the systematic behavior have largely been found to suffer from psychiatric illness, developmental disorders, cerebrovascular disease, dementia, head injuries, etc. Significant deviations from the normative data rarely occur in normal individuals.

The normative and/or clinical database may be a set of neurological records compiled from a large number of individuals, of various ages, who exhibit normal development and aging. The normative data may be used to identify the systematic changes within the healthy and normally functioning population. The systematic behavior of the normative data may be quantified to produce analyzing modules and corresponding correlation coefficients for patient diagnosis and/or analysis. The neurometric method and data correlations have been validated by research in more than a dozen countries, such as Barbados, China, Cuba, Germany, Japan, Korea, Mexico, the Netherlands, Sweden, Venezuela, and the United States and have been shown to be independent of culture and ethnicity. The reliability of these systematic changes has been extensively investigated and extremely high retest reproducibility has been demonstrated, indicating that the results are precisely predictable. The above-identified features demonstrate that neurometric analysis is a reliable and beneficial tool for neurological diagnosis.

A series of publications and patents in the name of Dr. E. Roy John relate to the field of EEG “neurometrics,” which is quantitative electrophysiological measurements (QEEG) evaluated relative to normative data. Generally, a subject's analog brain waves, at the microvolt level, are amplified, artifacts removed and the amplified brain waves convened to digital data. That data is then analyzed in a computer system to extract numerical descriptors which are compared to a set of norms (reference values), either the subject's own prior data (initial state) or a group of normal subjects of the same age (population norm). Such analyses can quantify the level, if any, of deviation of the activity of any brain region from the reference values.

One neurometric clinical quantitative EEG (QEEG) acquisition and analysis system is referred to as the Neurometric Analysis System (NAS) which is a proprietary system marketed by NxLink, Inc. The NAS is a system for QEEG analysis which has been made compatible with the formats of digital EEG apparatus produced by almost every major manufacturer of such equipment. Appropriate tests have been performed to confirm the accuracy of the interfaces which achieve compatibility with each device.

Currently, neurometric clinical tools such as NAS are utilized largely by neurological specialists such as neurologists or neurofeedback practitioners. The high costs of the NAS and similar systems have discouraged the use of neurometric analysis by others. For example, personal injury attorneys, airline companies, clinical psychologists, executive health services, military services, pediatricians, psychiatrists and school nurses are reluctant to use neurometric evaluations as they require these systems infrequently and can not justify the large purchase and maintenance costs.

### SUMMARY

The present invention relate to a system and method for screening patients and providing remote neurometric analysis and interpretive services for these analyses. Patients are screened away from neurological facilities by a portable neurometric device. Only patients that have been identified by the portable devices as requiring additional care may be referred to a neurologist. The system may include a remote facility with a remote server, a normative and/or clinical database and a plurality of neurometric analyzing modules. The normative and/or clinical database is quantified and systematic behaviors are extracted from the quantified normative and/or clinical database to generate analyzing modules, which include corresponding coefficients for the extracted numerical descriptors. The server may receive a request from the neurological facility or another user, via a communications network, for access to selected neurometric analyzing modules and their corresponding correlation coefficients. If the user is authorized, then in response to the user's request, the remote server permits access to the selected modules and corresponding correlation coefficients. The analyzing modules and corresponding correlation coefficients are applied to the digitized neurometric data to generate an output to the user. The output is reviewed and evaluated by the user and feedback may then be optionally is generated and sent back to the remote server for storage in the remote facility database. The optional feedback data may then be incorporated into the normative and/or clinical database to update and adjust the analyzing modules and corresponding correlation coefficients and thus, improve the quality of the outputs generated.

### BRIEF DESCRIPTION OF THE DRAWINGS

FIG. 1 is a diagram of an exemplary system of neurometric analysis according to an embodiment of the present invention; and

FIG. 2 is a diagram of an exemplary method of neurometric analysis according to one embodiment of the present invention.

### DETAILED DESCRIPTION

The present invention may be further understood with reference to the following description of preferred exemplary embodiments and the related appended drawings, wherein like elements are provided with the same reference numerals. It should be understood that although the preferred embodiments of the present invention will be described with reference to neurometric analysis based on the use of electroencephalograph (EEG) signals, the present invention may be implemented on a variety of neurometric information including, for example, Evoked Potentials, Event Related Potentials, Auditory Evoked Potentials, Topographical Brain Maps, Cognitive Evoked Potentials, Visual Evoke Potentials, Somatosensory Evoked Potentials, Brainstem Auditory Evoked Response as well as neuropsychological test results, etc.

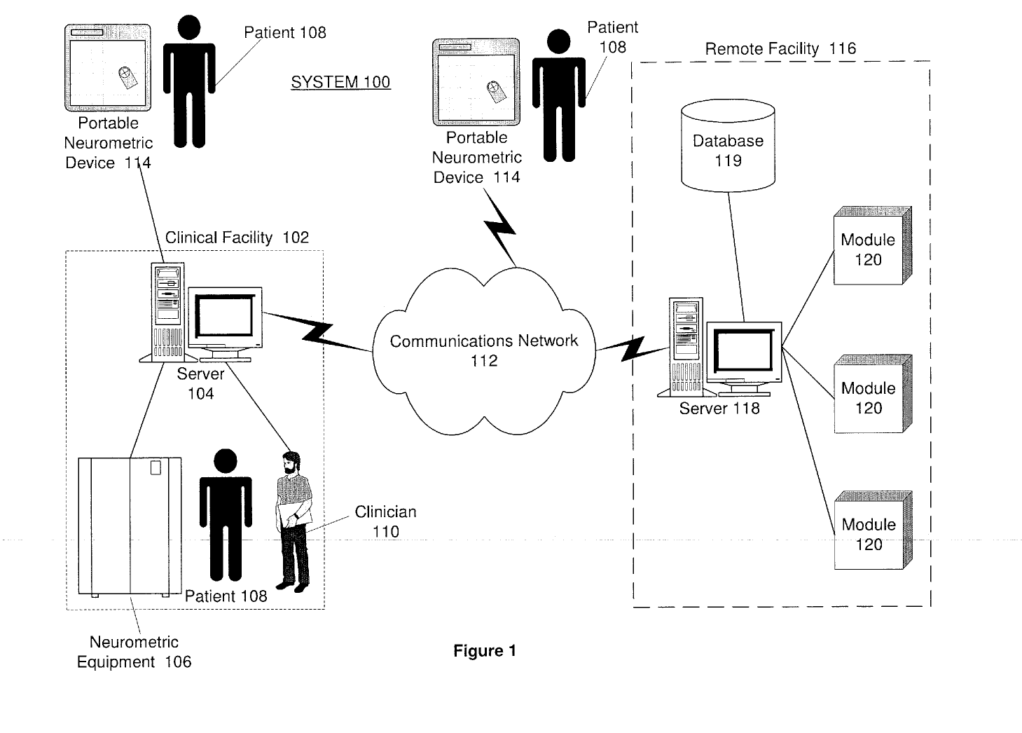

FIG. 1 is a diagram of an exemplary system of neurometric analysis according to one embodiment of the present invention. The system 100 may include one or more portable neurometric devices 114 or sets of devices utilized for collecting initial input data from a patient 108 and generating initial output data. The portable neurometric device 114 may then be connected to a local or remote computer to analyze the gathered data. For example, the portable device 114 may be connected to one or more neurological facilities 102 via a communications network 112, through a direct connection to the neurological facilities 102 or to a local computer which is remotely connected to a server via a network such as the Internet. In each of the models, the portable neurometric device 114 is preferably connected to a local or remote computer including software modules to perform all or a portion of the following functions:
1) reformatting of the digital EEG data (DEEG) into a standard neurometric format (NxEEG);
2) automatically edit and remove artifactual non-cerebral activity, usually detected as a statistical “non-stationarity” after calculating statistical descriptors of the EEG time series;
3) detect epileptiform activity to ensure that none of the activity identified as “artifacts” is actually pathophysiological—epileptic paroxysms and spikes are non-stationarities;
4) perform spectral or wavelet analysis to extract QEEG descriptors from the cleansed recording of, for example, about 48clean EEG segments each 2.5 seconds long;
5) perform neurometric analysis of the QEEG (NxQEEG), by transforming every numerical QEEG descriptor to a standard score or Z-score relative to the mean and standard deviation of that descriptor in the age-appropriate normative and/or clinical database, which is actually represented by a complex set of polynomial equations for each variable in every brain region (electrode channel);
6) evaluate the resulting matrix of about 2000 NxQEEG Z-scores by submitting the profile of abnormalities to a set of discriminant function, selecting and constraining the appropriate discriminant functions for relevance by consideration of a set of symptoms entered into a standardized clinical history format by the clinical facility at which the data are being collected;
7) scan the full NxQEEG matrix, the discriminant classification and patient history and automatically write an interpretative report, printing out graphic and numerical evidence computed from the raw EEG to support the interpretation; and
8) evaluate material condensed by the report writing module and recommend optimal substance and dosage for treatment of the patient taking all data into consideration as well as the clinical and pharmacological databases which are part of the IP downloaded to the remote terminal U.
9) compute three-dimensional QEEG source localization brain images based on the results which may be useful for therapeutic considerations and therapeutic monitoring.
   Additionally other non-pharmacological protocols based on the these findings may be offered such as neurofeedback, TMS, or other forms of stimulation therapies to alter deviant measures to bring them in line with expect normal range values. In addition, the system may require and indication by the subscriber of his/her acceptance of medical responsibility to validate the data.

For example, after initial evaluation using the portable neurometric device 114, the patient 108 may be referred to a neurological facility 102 for further examination and/or treatment. The neurological facility 102 may include one or more sources (e.g. neurometric equipment 106, neurologist 110, medical personnel, etc.) to further collect medical or neurological data from the patient 108 for neurometric analysis. Each source of additional data may be connected to one or more local servers 104, which are also connected to the communications network 112. A remote facility 116 serves as the central facility for maintaining and providing access to analyzing modules 120 for extensive neurometric analysis in the system 100. However, those skilled in the art will understand that the remote facility may make the modules for performing the above-listed functions available for execution on a local computer (e.g., via download) by providing access on a per-use basis or through any combination of the communications, data storage and processing capabilities of the remote facility 116 and the local computer. The remote facility 116 may be separate and independent from the neurological facilities 102 and may be located anywhere in the world. The remote facility 116 is connected to the communications network 112 and may, for example, include one or more servers 118 connected to one or more databases 119 where normative neurometric data may be stored. The normative neurometric data stored in the database 119 may be quantified and transformed to numerical descriptors. The quantified normative neurometric data may be utilized to generate and adjust the analyzing modules, which include corresponding correlation coefficients for the relevant numerical descriptors.

For example, in a first model, a clinician user (U) joins the system by accessing a Neurometric Network (NN) website as a subscriber and has a credit card or other payment source validated by NN. U identifies the manufacturer and model number of his DEEG apparatus (or this may be automatically detected when the device is connected) and downloads into the local computer or any computer controlling his DEEG desired ones of the separate software modules to perform any or all of the above-listed functions in the NxQEEG analysis listed above. The modules are disabled until receipt of a one-time use code as described below.

When after subscribing to NN, U desires to obtain an NxQEEG evaluation of a particular recording he has collected, he requests NN to enable the implementation of a subset or all of the NxQEEG modules to be applied to the particular case (e.g., identified by a unique number). Each module has a charge for a single utilization, known to the user and which may vary (e.g., depending on location, currency, etc). The NN website requests payment for the amount required to perform the requested operations and, upon receipt of authorization for that charge, downloads to U a code enabling his computer to activate the desired modules and perform the requested operations on the stored DEEG record.

Alternatively, the analysis modules may all reside at the NN server to which U uploads his digitized EEG record. U identifies the operations he would like performed on this data and a price is generated by the NN as described above. Upon receipt of the required payment, NN performs the analysis and sends the outputs numerical, graphic and report materials back to U via a network such as the Internet.

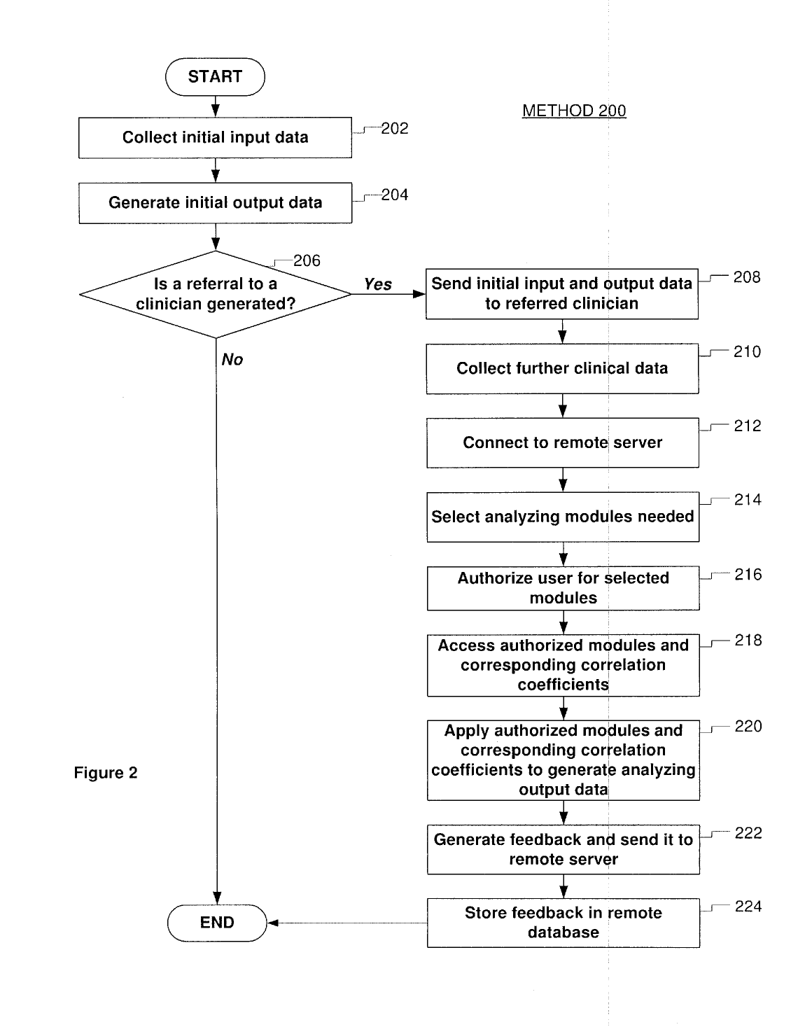

FIG. 2 shows an exemplary method 200 of neurometric analysis according to one embodiment of the present invention. In step 202, the portable neurometric device 114 or set of devices 114 collects initial input data from the patient 108. The initial input data may include records of neurological signals (e.g., EEGs) emitted by the patient's central nervous system, patient's response to medical procedures, patient's response to neurological procedures, patient's physical condition, patient's state of consciousness, patient's pre-existing conditions, patient's location, etc.

Subsequent to collecting the patient's initial input data 202, the portable neurometric device 114 generates a set of initial output data based the initial input data (step 204). An exemplary portable neurometric device may be, for example, the Brain Function Scan System disclosed in U.S. Pat. No. 6,052,619 by Erwin Roy John, who is also an inventor of the present invention. The disclosure of U.S. Pat. No. 6,052,619 is incorporated herein, by reference. However, those of skill in the art will understand that there may be other portable neurometric devices which may be utilized to collect initial input data from the patient 108.

The initial/output data may, for example, include a preliminary diagnosis, recommended procedures, recommended medications, recommended treatments, and/or predictions of treatment outcome. Furthermore, if the initial output data indicates that the patient requires additional care from a neurologist 110, a referral to a nearby neurological facility 102 may also be generated as part of the initial output data. In step 204, initial output data may be generated immediately following the collection of the initial input data 202. Alternatively, the initial input data may be stored and the initial output data may be generated at a later time.

A single portable neurometric device 114 may perform both functions of collecting initial input data and generating initial output data. However, the device for collecting initial input data need not be the same as the device for generating initial output data. Furthermore, the portable neurometric devices 114 may be, for example, mobile and onboard emergency vehicles such as ambulances or may be utilized in stationary facilities such as emergency rooms, clinics and/or physicians' offices.

The initial output generated from step 204 may be utilized as a screening tool to ensure that the patients 108 who require additional care from the neurologist 110 receive the appropriate treatment promptly. In step 206, the initial output data generated from step 204 is utilized to determine whether it is necessary for the patient 108 to pursue further medical and/or neurological treatment (e.g., whether the initial output data includes a referral to a local neurological facility 102). The neurological facility 102 may be, for example, a hospital, neurology clinic, physician's office, etc.

Step 206 serves as a screening process so that only those patients 108 who require the care of the neurologist 110 are referred. The present invention provides an efficient screening and access process for neurologists 110. All potential patients 108 may be screened and only those who require additional care and/or treatment may be further examined by the neurologists 110. Step 206 may help to eliminate unnecessary traffic to the neurologists 110 and allow them to focus on examining and treating those patients 108 who require their expertise. Furthermore, because the system 100 may eliminate unnecessary traffic to the neurologists 110, patients 108 who require the expert care of the neurologist 110 may be helped more readily.

Steps 202 and 204 may be performed regardless of the patient's location or state of consciousness. An advantage of the present invention, which utilizes portable neurometric devices 114, is the ability to analyze the patient 108 at any location and promptly refer the patient 108 to the neurological facility 102 for appropriate treatment when necessary. For example, an unconscious patient may be located in a rural area. The portable neurometric device 114 may provide an initial diagnosis of the patient 108 before he/she is transported to the neurological facility 102, which may be located miles away. The screening function of the present invention would prevent the patient 108 from unnecessarily traveling to the neurological facility 102, saving time and money. On the other hand, the initial diagnosis may promptly identify a condition requiring immediate care and refer the patient 108 to the neurologist 110 for appropriate testing and/or treatment in a rapid and efficient manner. The patient 108 would be referred to a nearby neurologist 110, minimizing the distance traveled.

As will be described in greater detail below, each neurological facility 102 may subscribe to any or all of various services provided by a remote facility 116 or may select the various software modules a la carte on a patient by patient basis. The subscribed services may not be the entire set of services available, i.e., for a facility catering to patients exhibiting specific pathologies only certain types of analysis of the EEG data may be required.

The portable neurometric device 114 may include the ability to receive and store messages from a neurological facility 102 or from the remote facility 116. The messages may include information such as the list of subscribed services for a variety of neurological facilities 102. Thus, the portable neurometric device 114 may identify an appropriate neurological facility 102 to which the patient should be referred.

If the initial output data indicates that the patient 108 requires additional care by the neurologist 110, a referral to the local neurological facility 102 is generated and the initial input and output data are transmitted to the referred neurological facility (step 208). The data may be transmitted over a communications network, wired or wireless, such as, for example, the Internet, Wide-area network (WAN), Satellite network, Cellular network, etc. When transmitting the initial input and output data over a public or shared communications network 112, the data may be protected by security and privacy protocols such as encryption as would be understood by those skilled in the art. The initial input and output data may alternatively be transmitted using a direct connection, wired or wireless, to the neurological facility such as a cradle, ethernet port, telephone jack, USB port, etc.

In step 210, further medical and/or neurometric data may be collected at the neurological facility 102. As will be explained in greater detail below, the additional data collected along with the initial output data from the portable neurometric device 114 may become part of the analyzing input data used for complete/extensive neurometric analysis. The additional data may be collected, for example, by medical procedures, neurometric procedures, interviews with medical staff, obtaining previous medical records and/or examinations by the neurologist 110. For example, the neurological facility 102 may have an EEG recording neurological signals emitted from the patient 108. The neurologist 110 and/or medical personnel may also collect analyzing input data by examining the patient 108 and/or interviewing the patient 108 for additional information, such as his/her medical history. In addition, the neurologist 102 may review and add the initial input and output data to the analyzing input data for complete/extensive neurometric analysis.

The input data collected from various sources within the neurological facility 102 are forwarded to the local server 104 or to any other local computer. In step 212, the local server is connected to the remote server 118 via the communications network 112 and the user identifies to the remote server 118 the analyzing modules 120 to be used. The analyzing modules 120 to be used may be selected, as would be understood by those skilled in the art, as a function of the input data or other circumstances unique to the patient either automatically software or by the neurologist 110 either on his own judgement or assisted by a preliminary analysis of the EEG data (step 214). The neurologist 110 may review all the analyzing input data and make a preliminary determination that the patient may exhibit one or more of certain neurological conditions for which neurometric data has been compiled such as, for example, Depression, Bipolar Disorder, Alcoholism, Dementia, Schizophrenia, Learning Disability, Attention Deficit Disorder (ADD), Attention Deficit/Hyperactivity Disorder (ADHD), Maturational Lag, Development Deviation, Head Injury, Obsessive Compulsive Disorder, Ritalin response, Cerebrovascular Obstruction (stroke), Hemorrhage (bleeding), Brain Damage, Brain Dysfunction, Ischemic Stroke (“brain attack”), Spinal Injury, Coma, Death, etc. Based on this preliminary determination the neurologist 110 (or other health professional) may select desired ones of the software analyzing modules 120 for use.

Once the neurologist 110 has reviewed any software recommendations and selected a desired set of analyzing modules 120, the software may respond to the selection of modules 120 and recommend a more appropriate set of modules 120. For example, upon review of the input data the neurologist 110 may select ADHD as a condition which utilizes particular modules 120. The computing device may, however, disagree and alert the neurologist 110 that the selection is considered potentially inappropriate and recommend other conditions and modules 120, e.g., ADD, in place of the initial selection. In a further alternative embodiment, the neurologist 110 may manually select a set of modules 120 for analysis. As described above, the software for the various modules may reside on the local server 104 or other local computer or it may reside on any other computer (e.g., the remote facility server 118) connected to the local server 104 via a network (e.g., the neurologist's personal computer, dedicated computing devices, neurometric equipment 106, etc.).

As described above, each of the analyzing modules 120 performs a designated task for analyzing the neurometric data. For example, a first module 120 preferably reformats the raw digital EEG data (DEEG) into a standard neurometric format (NxEEG) and a second module 120 edits this NxEEG data to remove artifactual non-cerebral activity, usually detected as a statistical “non-stationarity” after calculating statistical descriptors of the EEG time series. A third module 120 detects epileptiform activity to ensure that none of the activity identified as “artifacts” is actually pathophysiological—epileptic paroxysms and spikes are classified as non-stationarities. A fourth module 120 performs spectral or wavelet analysis to extract QEEG descriptors from the cleansed recording of, for example, about 48clean EEG segments each 2.5 seconds long and a fifth module 120 performs neurometric analysis of the QEEG (NxQEEG), by transforming every numerical QEEG descriptor to a standard score or Z-score relative to the mean and standard deviation of that descriptor in the age-appropriate normative and/or clinical database, which is actually represented by a complex set of polynomial equations for each variable in every brain region (electrode channel). Thereafter, a sixth module 120 evaluates the resulting matrix of about 2000 NxQEEG Z-scores by submitting the profile of abnormalities to a set of discriminant function, selecting and constraining the appropriate discriminant functions for relevance by consideration of a set of symptoms entered into a standardized clinical history format by the clinical facility at which the data are being collected and a seventh module 120 scans the full NxQEEG matrix, the discriminant classification and patient history and automatically prepares an interpretative report, printing out graphic and numerical evidence computed from the raw EEG to support the interpretation. Finally, an eighth module 120 evaluates material condensed by the report writing module and recommends a course of treatment (e.g., optimal substance and dosage of therapeutic agents) for the patient taking all data into consideration as well as the clinical and pharmacological databases which are part of the information downloaded to the remote terminal U. Thereafter, a ninth module 120 computes three-dimensional QEEG source localization brain images based on the results. The ninth module 120 may, for example, be a Low or Variable Resolution Electromagnetic Tomography (Loreta/Vareta) module as would be understood by those skilled in the art. Those skilled in the art will further understand that a wide variety of additional software modules may be made available as well including, for example, a Univariate Feature Extraction module, a Multivariate Feature Computation module, a Discriminant Clinical Classification of Profile module, a module for Cluster Analysis and Subtyping to Predict Response to Treatment or Evolution of Illness, a Numerical Tabular Outputs module, a Topographic Maps module for quantitative QEEG topographic mapping, a clinical performance or human enhancements correlates module, etc.

As described above, access to analyzing modules 120 and corresponding correlation coefficients, is preferably restricted to authorized users. In step 216, a user may become authorized for access to the selected analyzing modules 120 and corresponding correlation coefficients. The remote server 118 may, for example, require users to provide subscription codes or payment prior to accessing and using the analyzing modules 120. Users, for example, may provide payment and become authorized based on, for example, an unlimited monthly use basis for all modules, unlimited monthly use basis for selected modules, monthly quota basis for all modules, monthly quota basis for selected modules, per use basis for each individual module, etc. Subscriptions and/or payment may be, for example, provided by credit card, monthly bills, and/or prepaid fees.

The remote facility 116 serves as the central facility for maintaining, updating and providing access to analyzing modules 120 and alleviating neurological facilities 102 or any authorized user from the burden of purchasing and maintaining the equipment and tools necessary for conducting neurometric analysis. Nonetheless, the analyzing modules 120 remain available to authorized users when needed (step 218). The reduced cost for neurometric analysis in the present invention may permit users other than neurology specialists (e.g., neurologists 110, neurofeedback practitioners, etc.) to afford and utilize this tool. Some of these other users may include personal injury attorneys, airline companies, clinical psychologists, executive health services, military services, pediatricians, psychiatrists, school nurses, etc.

Thus, upon authorization, the user may apply the selected and authorized modules 120 and corresponding correlation coefficients to the analyzing input data to generate analyzing output data (step 220). Analyzing output data generated may include diagnosis, predictions, suggested treatment, etc. Neurologists 110 may utilize the analyzing output data generated as a guideline and tool for further diagnosis and treatment.

After the analyzing output data has been generated, the neurologist 110 may review the analyzing output data and compare the analyzing output data to the initial output data, the input data collected at the facility 102, the neurologist's own diagnosis, etc. Based on these comparisons, feedback data is generated and transmitted to the remote server (step 222). The feedback data may be, for example, quantified amounts of deviation from the neurologist's diagnosis, deviation of the patient's behavior, deviation from the initial input and output data, etc. The feedback data may then be incorporated into the data in the system 100 to improve the analyzing output data generated. For example, a certain level or type of neurological activity may not have been previously associated with a certain condition.

Upon receipt by the remote server, the feedback data is stored in a remote database (step 224). Based on the feedback, the system 100 will “learn” from each patient 108 processed. The normative and/or clinical database 119, the analyzing modules 120 and the corresponding correlation coefficients may be modified to more accurately represent the systematic behavior of the general normal population or a particular non-normal population. For example, the normative and/or clinical database 119, may be expanded and/or updated by the feedback data. New numerical descriptors may be generated as a function of the modified normative data stored in the database 119. Systematic changes may be re-extrapolated from the modified normative data and new numerical descriptors to generate updated correlation coefficients. Thus, the analyzing modules adjust based on the feedback data to improve the quality of the output data generated.

In the example stated above, the level/type of neurological activity may be associated with the condition exhibited by the patient. Those of skill in the art will understand that modules may be changed based on a single patient, but it is more likely for modules to change based on data from a statistically significant sampling.

The interface between the system 100 and the users may be represented in various languages. Because the communications network 112 may have an international reach, the multi-language interface can enable the use of this system internationally, particularly in non-English speaking nations. The multi-language interface may expand the user base of the system 100 beyond the United States and to other countries, for example, European and Asian countries, many of which have widespread awareness and acceptance of neurometrics.

While specific embodiments of the invention have been illustrated and described herein, it is realized that numerous modifications and changes will occur to those skilled in the art. It is therefore to be understood that the appended claims are intended to cover all such modifications and changes as fall within the true spirit and scope of the invention.

What is claimed is:

1. A system, comprising:
a plurality of neurometric analyzing software modules operating on electroencephalogram (EEG) data, each module performing a defined task with respect to neurometric analysis of the EEG data;
a database including normative neurometric data;
a server including an authorization module, the authorization module comparing data received from a remote user to determine whether the user is authorized to utilize any of the plurality of analyzing software modules
an analysis module configured to receive input data from a patient and select one of the neurometric analyzing modules based on the input data, wherein the selected one of the neurometric analyzing modules compares the input data to the normative neurometric data to generate an output, the analysis module selecting the one of neurometric analyzing modules from a set of subscribed neurometric analyzing modules.

2. The system of claim 1, wherein the normative neurometric data includes a plurality of historic neurological records.

3. The system of claim 1, wherein the normative neurometric data is quantified.

4. The system of claim 1, wherein the normative neurometric data includes historical records of electroencephalograph (EEG) signals.

5. The system of claim 1, further comprising:
at least one neurological facility connected to a remote facility via a communications network, wherein the remote facility includes the neurometric analyzing modules, the database and the analysis module.

6. The system of claim 5, wherein the remote facility is separate and independent from the neurological facility.

7. The system of claim 5, wherein the remote facility further includes a server which controls all data traffic to and from the remote facility

8. The system of claim 5, further comprising:
at least one portable neurometric device configured to pre-screen the patient, generate a preliminary diagnosis and refer the patient to the neurological facility, when necessary.

9. The system of claim 5, wherein the neurological facility includes at least one of a neurometric equipment, a neurologist and medical personnel.

10. A method for neurometric analysis, comprising the steps of:
obtaining preliminary input data from a patient;
generating a preliminary diagnosis as a function of the preliminary input data, wherein the patient is pre-screened based on the preliminary diagnosis to determine if a referral to a neurological facility is necessary; and
referring the patient to the neurological facility, if the preliminary diagnosis indicated that the referral was necessary.

11. The method of claim 10, further comprising the steps of:
obtaining further input data from the referred patient; and
generating an output as a function of at least one of the preliminary input data, the preliminary diagnosis, and the further input data.

12. The method of claim 11, wherein the further input data includes a neurologist's diagnosis.

13. The method of claim 10, further comprising the steps of:
selecting a neurometric analyzing module from a plurality of neurometric analyzing modules; and
generating an output by comparing at least one of the preliminary input data, the preliminary diagnosis, and further input data to normative neurometric data using the selected neurometric analyzing module.

14. The method of claim 13, wherein the normative neurometric data includes a plurality of historic neurological records.

15. The method of claim 13, wherein the normative neurometric data is quantified.

16. The method of claim 13, wherein the normative neurometric data includes historical records of electroencephalograph (EEG) signals.

17. The method of claim 11, further comprising the steps of:
generating feedback data, wherein the feedback data includes suggested improvements to the analyzing modules; and
updating the normative neurometric data and the analyzing modules based on the feedback data.

18. A portable neurometric device comprising:
a recording element configured to obtain preliminary input data from a patient; and
a diagnostic module configured to generate a preliminary diagnosis as a function of the preliminary input data, wherein the patient is pre-screened based on the preliminary diagnosis to determine if a referral to a neurological facility is necessary.

19. The portable neurometric device of claim 18, wherein the receiving element includes an EEG recording element configured to record EEG signals of a patient's head.

20. The portable neurometric device of claim 19, wherein the EEG signals are recorded in digital form.

21. The portable neurometric device of claim 19, wherein the receiving element further includes a quantifying element configured to convert EEG signals to numerical descriptors.

22. The portable neurometric device of claim 18, wherein the diagnostic module may further generate a referral to a nearby neurological facility as a function of the preliminary diagnosis and a subscription of neurometric analyzing modules by the neurological facility.

23. The portable neurometric device of claim 18 further comprising a communications element connected to a communications network and configured to transmit, receive, and store messages.

24. The portable neurometric device of claim 23 wherein the messages include subscription data of neurometric analyzing modules by the neurological facility.

25. The portable neurometric device of claim 23 wherein the communications element is configured to transmit the preliminary input data to the referred neurological facility

26. The portable neurometric device of claim 25 wherein the communication element is further configured to transmit data by security protocols.
